(19) United States
(12) Patent Application Publication
(10) Pub. No.: US 2019/0198180 A1
(43) Pub. Date: Jun. 27, 2019

(54) Automatic Visual Recognition Intravenous Infusion Drop Counting Application

(71) Applicant: Alejandro C. Cervantes, San Diego, CA (US)

(72) Inventor: Alejandro C. Cervantes, San Diego, CA (US)

(21) Appl. No.: 15/853,797

(22) Filed: Dec. 24, 2017

(51) Int. Cl. G16H 80/00; G16H 40/67; G16H 30/40; G06K 9/00; A61M 5/168

(52) U.S. Cl. CPC G16H 80/00; G16H 40/67; G16H 30/40; A61M 2205/505; A61M 5/1689; A61M 2205/3306; A61M 2205/3592; G06K 9/00671

(57) ABSTRACT

An automated system, method or computer program product for providing visual recognition and counting of drops falling into a drip chamber for intravenous therapies (drip rate). The Automatic Visual Recognition Intravenous Infusion Drop Counting Application software subroutine framework can run on a mobile device and can be configured to, when activated, calculate and visually recognize the drip rate of intravenous therapies. The functionality of the Automatic Visual Recognition Intravenous Infusion Drop Counting Application software subroutine framework allows the user to manage standard drip sets. Additionally, different drip set values can be assigned. The Automatic Visual Recognition Intravenous Infusion Drop Counting Application software subroutine framework also allows the user to manage the infusion; application can integrate a timer and other diverse features. The updated information can be stored in the internal memory of the device, other information recording medium, a memory data storage device or into a web based storage service.

### BACKGROUND OF THE INVENTION

The present invention relates to the field of healthcare related applications, and in particular, applications for mobile computing devices such as smartphones and tablets.

When a doctor gives an order to a nurse to administer an infusion, there are different scenarios in which the nurse will perform the infusion, in a fully modern hospital, the hospital system will calculate the drip rate of the infusion and usually, an infusion pump will administer the infusion. Therefore, the nurse does not have to calculate de drip rate and set up the drip rate in the infusion pump. However, in a scenario where the nurse has to calculate the drip rate manually and has to use a gravity drip set for intravenous infusion, the nurse has to, manually, set up the drip rate.

The usual way to check an intravenous infusion drip rate is as follows the nurse counts the drops passing through the intravenous drip chamber for a fifteen seconds lapse, and then she multiplies this value by four, and this is the average drip rate of the infusion and will always be an even number. If the current drip rate is thirty-two drops per minute and the desired drip rate is sixty-five drops per minute, the nurse has to open the valve to increase the drop flow. Then she has to repeat all the process; count for fifteen seconds the drops passing through the drip chamber and multiply it by four and that will be the new current drip rate, if the result is fifty-six drops per minute, for example, then the nurse has to do the process all over again. As we can see, the process is tedious and inaccurate.

With the Automatic Visual Recognition Intravenous Infusion Drop Counting Application software subroutine framework, it can be much faster, easier and accurate: the nurse only has to point the camera to the falling drops into drip chamber and this application will detect the drip rate and displaying it in the screen of the mobile device. If it is lower or higher, the nurse can adjust the valve until the application displays the correct drip rate. The Automatic Visual Recognition Intravenous Infusion Drop Counting Application software subroutine framework can performance this routine in a much faster and easier way; the nurse only has to point the camera to the drip chamber and the application will detect the drip rate until it matches the required value.

### SUMMARY OF THE INVENTION

The Automatic Visual Recognition Intravenous Infusion Drop Counting Application software subroutine framework is an integration of image recognition algorithms, object detection algorithms and computer program instructions to, automatically, detect, count, and calculate the drip rate of, the falling drops into a drip chamber; this feature improves the determination of the drip rate.

The Automatic Visual Recognition Intravenous Infusion Drop Counting Application software subroutine framework is a component of a method of interaction; this process runs on a mobile phone that connects to a remote server containing data that either stores the data or processes it to be used on the device, making the device function in a determined way.

The Automatic Visual Recognition Intravenous Infusion Drop Counting Application also allows the nurse to manage many of the different steps or aspects that are involved in the process to administer an intravenous infusion. These aspects include, among others, in an illustrative but not limitative list: patient information or as to whom the intravenous infusion needs to be administered, what is to be administered in the infusion, the calculation of the drip rate of the infusion, setting up the infusion to the drip rate calculated, duration of the infusion, track and schedule of the number of infusions prescribed to the patient, notification to the nurse when the infusion is about to be completed, keep a record of the infusions given to a patient.

### DETAILED DESCRIPTION

The following is a detailed description of exemplary embodiments to illustrate the principles of the invention. The embodiments are provided to illustrate aspects of the invention, but the invention is not limited to any embodiment. The scope of the invention encompasses numerous alternatives, modifications and equivalent; it is limited only by the claims.

Numerous specific details are set forth in the following description in order to provide a thorough understanding of the invention. However, the invention may be practiced according to the claims without some or all of these specific details. For the purpose of clarity, technical material that is known in the technical fields related to the invention has not been described in detail so that the invention is not unnecessarily obscured.

A invention 304, that detects falling drops into a drip chamber 202; and, automatically, starts counting the drops 204 to show it in a display window 200; and, furthermore, compares the drip rate value obtained from the user's calculations and the physical drip rate occurring into the drip chamber into an interactive user interface 104; and furthermore, notifying the user when the required drip value is achieved 416.

The advantages of the present invention include, without limitation, an improvement in the accuracy of the drop counting for the manual set up of intravenous infusion therapies, as shown in FIGS. 6-600, a reduction of the required time that a user needs to check the drip rates values, an enhancement of the visual capability of the medical staff in charge of the administration of intravenous infusion therapies, an improvement in the nurse activities to prioritize the needing of each patient, the capability to keep a record of the therapies administered to each patient, an instantaneous nurse's backup of each therapy infusion without the limitation to be writing down in paper all the aspects involved in an intravenous infusion therapy; furthermore, the invention can be linked to a central hospital's server to automatically store the selected data.

As will be appreciated by one skilled in the art, aspects of the present invention may be embodied as a system, method or computer program product. Accordingly, aspects of the present invention may take the form of an entirely hardware embodiment, an entirely software embodiment (including firmware, resident software, micro-code, etc.) or an embodiment combining software and hardware aspects that may all generally be referred to herein as a “circuit,” “module” or “system.” Furthermore, aspects of the present invention may take the form of a computer program product embodied in one or more computer readable medium(s) having computer readable program code embodied thereon.

Any combination of one or more computer readable medium(s) may be utilized. The computer readable medium may be a computer readable signal medium or a computer readable storage medium. A computer readable storage medium may be, for example, but not limited to, an electronic, magnetic, optical, electromagnetic, infrared, or semiconductor system, apparatus, or device, or any suitable combination of the foregoing. More specific examples (a non-exhaustive list) of the computer readable storage medium would include the following: an electrical connection having one or more wires, a portable computer diskette, a hard disk, a random access memory (RAM), a read-only memory (ROM), an erasable programmable read-only memory (EPROM or Flash memory), an optical fiber, a portable compact disc read-only memory (CD-ROM), an optical storage device, a magnetic storage device, or any suitable combination of the foregoing. In the context of this document, a computer readable storage medium may be any tangible medium that can contain, or store a program for use by or in connection with an instruction execution system, apparatus, or device.

A computer readable signal medium may include a propagated data signal with computer readable program code embodied therein, for example, in baseband or as part of a carrier wave. Such a propagated signal may take any of a variety of forms, including, but not limited to, electro-magnetic, optical, or any suitable combination thereof. A computer readable signal medium may be any computer readable medium that is not a computer readable storage medium and that can communicate, propagate, or transport a program for use by or in connection with an instruction execution system, apparatus, or device.

Program code embodied on a computer readable medium may be transmitted using any appropriate medium, including but not limited to wireless, wireline, optical fiber cable, RF, etc., or any suitable combination of the foregoing. Computer program code for carrying out operations for aspects of the present invention may be written in any combination of one or more programming languages, including an object oriented programming language such as Java, Smalltalk, C++ or the like and conventional procedural programming languages, such as the “C” programming language or similar programming languages. The program code may execute entirely on the user's computer, partly on the user's computer, as a stand-alone software package, partly on the user's computer and partly on a remote computer or entirely on the remote computer or server. In the latter scenario, the remote computer may be connected to the user's computer through any type of network, including a local area network (LAN) or a wide area network (WAN), or the connection may be made to an external computer (for example, through the Internet using an Internet Service Provider).

Aspects of the present invention are described below with reference to flowchart illustrations and/or block diagrams of methods, apparatus (systems) and computer program products according to embodiments of the invention. It will be understood that each block of the flowchart illustrations and/or block diagrams, and combinations of blocks in the flowchart illustrations and/or block diagrams, can be implemented by computer program instructions. These computer program instructions may be provided to a processor of a general purpose computer, special purpose computer, or other programmable data processing apparatus to produce a machine, such that the instructions, which execute via the processor of the computer or other programmable data processing apparatus, create means for implementing the functions/acts specified in the flowchart and/or block diagram block or blocks.

These computer program instructions may also be stored in a computer readable medium that can direct a computer, other programmable data processing apparatus, or other devices to function in a particular manner, such that the instructions stored in the computer readable medium produce an article of manufacture including instructions which implement the function/act specified in the flowchart and/or block diagram block or blocks.

The computer program instructions may also be loaded onto a computer, other programmable data processing apparatus, or other devices to cause a series of operational steps to be performed on the computer, other programmable apparatus or other devices to produce a computer implemented process such that the instructions which execute on the computer or other programmable apparatus provide processes for implementing the functions/acts specified in the flowchart and/or block diagram block or blocks.

FIG. 1 is an embodiment of the invention 304, showing a mobile device 100 graphical user interface (GUI) 308 illustrating a sample implementation of the invention 104. In accordance with embodiments of the inventive arrangements disclosed herein. The Visual Recognition and Automatic intravenous Infusion Drop Counting Application 304 is a mobile application with processes 104, 106, 108, 110, where images 104 through 110 are for illustrative purposes only, and are not meant to be interpreted as an absolute implementation or limitation of an embodiment of the present disclosure.

A mobile device 100, for example an iPHONE, BLACKBERRY or ANDROID smartphone, can provide access to the application. The mobile device 100 can include a display area 112 and an input mechanism 114, which, in this example, are one-and-the-same. That is, the display area 112 of a mobile device 100 can also be used as the input mechanism 114, for example, with a touch screen.

Into the environment of a mobile application framework 104, a user (nurse) 300 aims a drip chamber 202 with the camera 102 incorporated into the mobile device 100, and the invention 304 determines when a falling drop is detected 108 into the drip chamber 202 and starts comparing the result from a previously calculated drip rate 106 and the actual value of the drip rate occurring 110 into the drip chamber 202.

FIG. 2 illustrates the practical use of the invention, showing a interactive user interface 200 from the invention 304, when said interactive user interface 200 is triggered 310 the falling drops are detected into the drip chamber 202 and its drip rate is displayed to the user (nurse) 300 with the simultaneous detection and counting feature 204 of the invention 304.

FIG. 3 is a schematic diagram illustrating the invention functionality; it shows the stages of the process of this invention, a user 300 with a mobile device 302, which usually incorporates at least one camera 102, and a storage medium 306, stores this process 304 to run it into the mobile device 302 that connects to a remote server 314. The user 300 starts using the invention 304 by interaction mechanisms (Graphical user interfaces) 308. When the user 300 activates the process 304 to check the drip rate of an intravenous infusion, the graphical user interfaces 308 configuration guides the user to fill the intravenous infusion therapy required data 306 to start calculation and to allow the user to trigger 310 the visual recognition framework 104. This data can be stored directly into the mobile device 302 storage medium 306, sent through a network 312 to the application's server 314, in which the user can manage its own patient's profiles stored 316; or any other server, like a hospital's server 318.

Additionally, in this instance, the user can activate a reminder to be notified when the intravenous infusion therapy is going to end.

The invention 304 can include one or more graphical user interfaces (GUIs) 308, and a trigger user interface 310 and an interactive user interface 200 comprising framework 104 with 106 and 110 outputs and the image recognition mechanism 108. A GUI 308 can represent the interaction mechanism by which a user can perform functions with and/or enter data into the application 304. Multiple GUIs 308 can exist, each presenting different data and/or functionality. The quantity of GUIs 308 and/or their configuration can depend upon the design and implementation of application 304.

Network 312, can include any hardware/software/and firmware necessary to convey data encoded within carrier waves. Data can be contained within analog or digital signals and conveyed though data or voice channels. Network 312, can include local components and data pathways necessary for communications to be exchanged among computing device components and between integrated device components and peripheral devices. Network 312, can also include network equipment, such as routers, data lines, hubs, and intermediary servers, which together form a data network, such as the Internet. Network 312, can also include circuit-based communication components and mobile communication components, such as telephony switches, modems, cellular communication towers, and the like. Network 312, can include line based and/or wireless communication pathways.

As used herein, presented data stores 306, 314 and 318 can be a physical or virtual storage space configured to store digital information. Data stores 306, 314 and 318 can be physically implemented within any type of hardware including, but not limited to, a magnetic disk, an optical disk, a semiconductor memory, a digitally encoded plastic memory, a holographic memory, or any other recording medium. Data stores 306, 314 and 318 can be a stand-alone storage unit as well as a storage unit formed from a plurality of physical devices. Additionally, information can be stored within data stores 306, 314 and 318 in a variety of manners. For example, information can be stored within a database structure or can be stored within one or more files of a file storage system, where each file may or may not be indexed for information searching purposes. Further, data stores 306 and/or 314 and/or 318 can utilize one or more encryption mechanisms to protect stored information from unauthorized access.

FIG. 4 is a flowchart of the invention 304, for improving the administration of intravenous infusion therapies. The user 300 initializes the main application 400, then, when the user 300 has finished to fill the required data for calculation of the drip rate 106, then is allowed to start the visual recognition framework 104, this also initializes the image recognition algorithms 402 in searching for predetermined frames and movement parameters 404, which are part of the computer program code of the invention 304. The invention 304 establish a timeout to perform this search, if a falling drop is not detected into the timing parameters 406, the application send a message to the user to make a new search 408 and returns to step 404, is a falling drop is detected the application automatically starts counting 410 and then comparing 412 the occurring drip rate into the drip chamber 202 versus the calculated (required) drip rate 106, this information is displayed into the interactive user interface 200 from the invention 304, and this interaction will keep going until both drip rate values matches 106, 110, allowing the user 300 to adjust 414 the valve mechanism 500 of the therapy and meet the desired value in an most faster and accurate way. When both values are equal, the invention 304 shows a message 416 to the user 300, then the next step of the process 304 is to allow the user 300 to fix the flow rate and manage the information of this intravenous infusion therapy 418, additionally creating a backup file 420, to be stored either in the remote server of the application 314 or in the internal storage medium 306 of the mobile device 302. This last feature allows the nurse to maintain focus on the patient.

FIG. 5 is an illustration of a valve mechanism commonly used to set up the drip, this kind of mechanism is what a nurse 300 always needs to adjust manually 414, when an infusion pump is not available to make an intravenous infusion therapy.

FIG. 6 illustrates the usual way to check the drip rate, in this FIG. 600, a user 300 is using a watch 602 to perform an average measurement of the drip rate, note an advantage of the invention 304 is that the user/nurse does not need to start the counting process all over again each fifteen seconds, because the invention 304 keeps continuous tracking 104 of the occurring drip rate into the drip chamber 202, making an accurate measurement of the drip rate in even less than fifteen seconds, the nurse 300 is always looking at the drip chamber 202, and can adjust the valve mechanism 604 simultaneously, because the application 304 shows the occurring drip rate in real time.

### BRIEF DESCRIPTION OF THE DRAWINGS

FIG. 1 is an embodiment of the invention

FIG. 2 illustrates the practical use of the invention

FIG. 3 is a schematic diagram illustrating the invention functionality

FIG. 4 is a flowchart of the invention functionality

FIG. 5 is an illustration of a valve mechanism used to set up the drip rate

FIG. 6 illustrates a usual way to check the drip rate

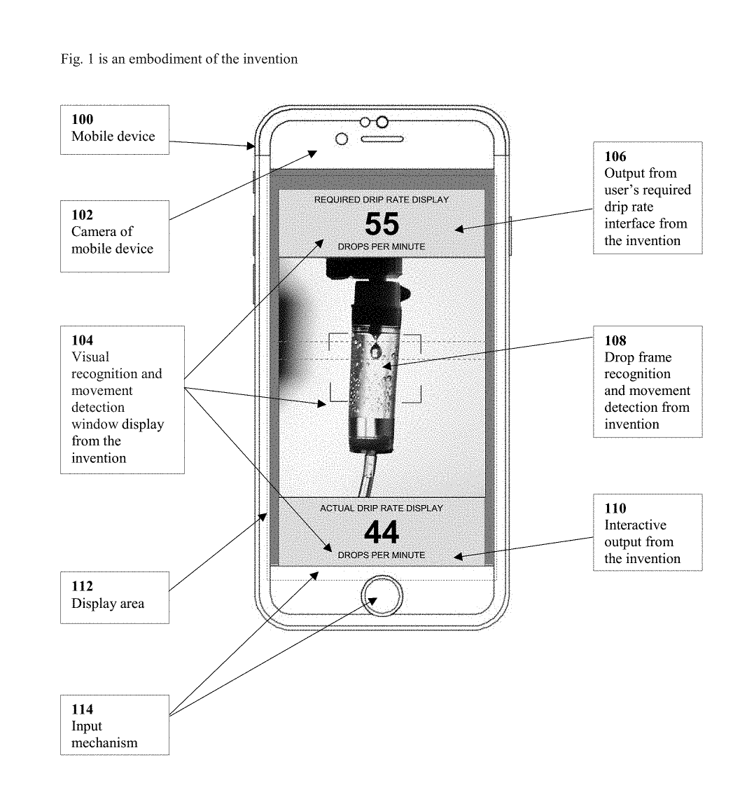

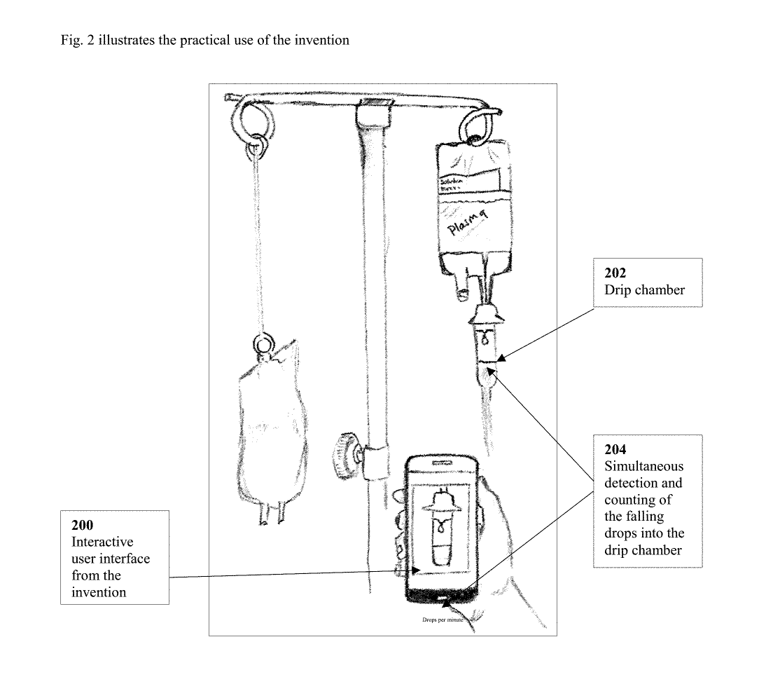

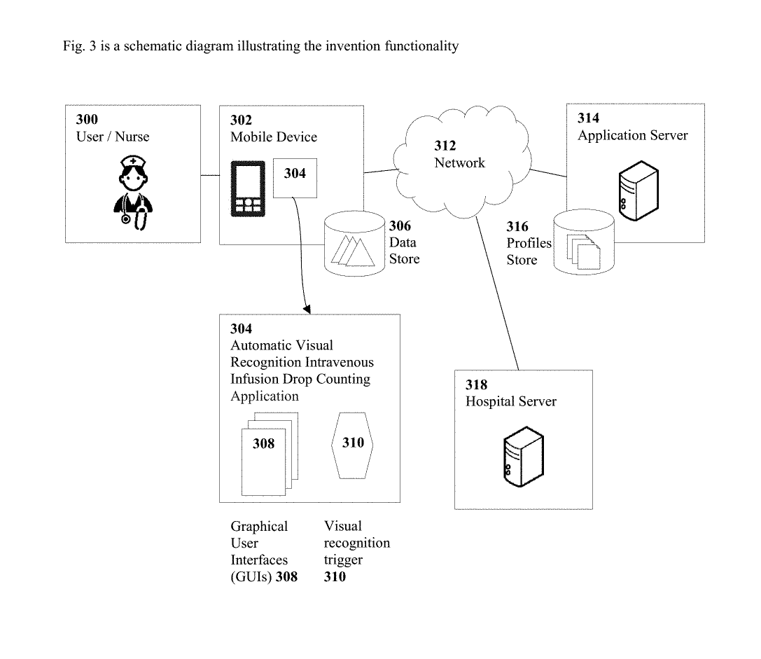

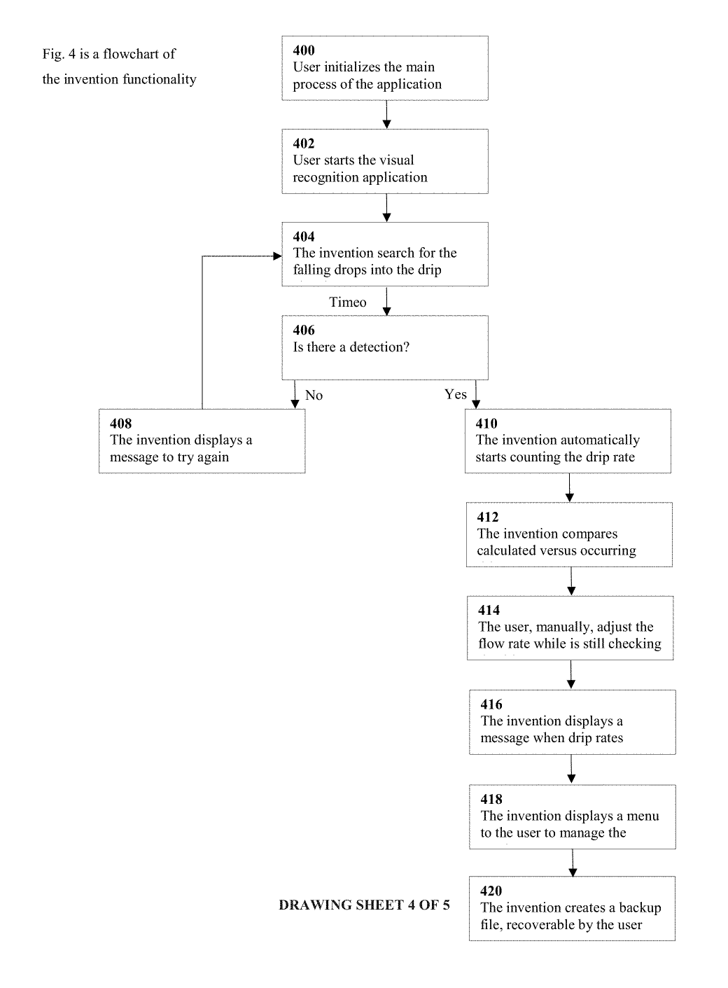

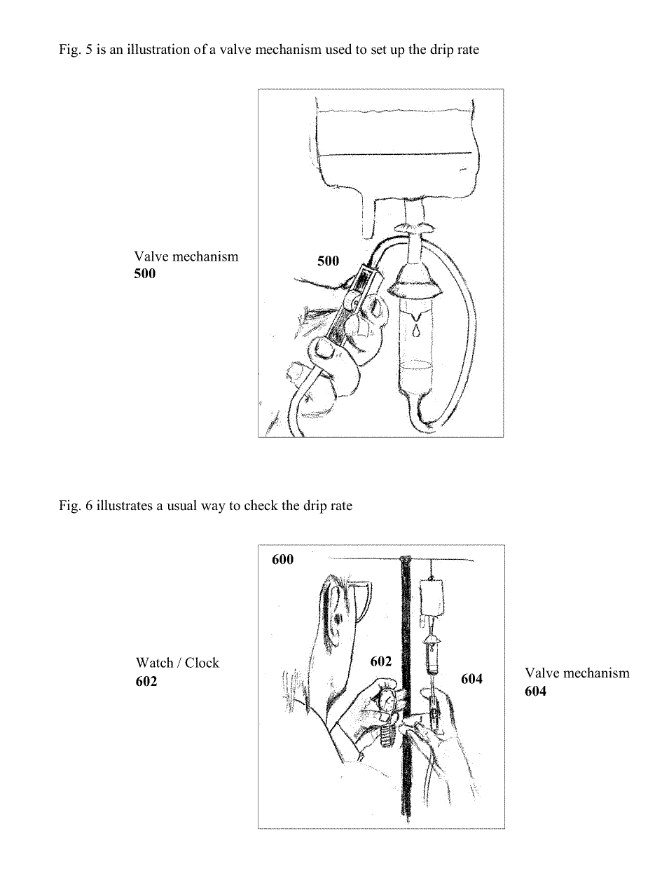

The invention what is claimed is:

1. A method in a mobile device comprising:
receiving at a mobile device a visual triggering input for a drip rate calculation framework application, wherein the application runs on the mobile device and facilitates the administration and accuracy of an intravenous infusion therapy.
determining the actual drip rate occurring into a drip chamber of a drip set;
capturing visual information to display drip rate value;
automatically showing the visual input values versus a required drip rate;
determining whether or not the actual drip rate matches the required drip rate value;
responsively recognizing the drops falling into a drip chamber; and
continuing checking the physical drip rate until the required drip rate is achieved.

2. The method of claim 1, wherein and alarm and a visual signal are activated when the calculated drip rate matches the visually recognized drip rate.

3. The method of claim 1, wherein the visual recognition allows the user to check the drip rate continuously until the average value and the calculated value are synchronized

4. The method of claim 1 further comprising:
displaying a configuration wizard interface upon initial activation of the Automatic Visual Recognition Intravenous Infusion Drop Counting Application software subroutine framework, wherein the configuration wizard interface consists of configuration GUIs and input data GUIs.

5. The method of claim 1, further comprising:
detecting the falling of solution drops into a drip chamber and displaying the occurring the drip rate.

6. A method of claim 1, further comprising:
a interface of the camera of the mobile device upon which Automatic Visual Recognition Intravenous Infusion Drop Counting Application software subroutine framework runs receiving visual input, said visual input comprising movement detection, of a drop into the drip chamber, from an image or video received from a camera;
automatically determining from visual input, an existence of a falling drop into the drip chamber; and
responsive to the determining, automatically providing the actual drip rate occurring into the drip chamber

7. A computer program product, the computer program product comprising a non-transitory computer readable storage medium having computer usable program code embodied therewith, the computer usable program code comprising:
computer usable program code configured to receive at a mobile device a visual input for Automatic Visual Recognition Intravenous Infusion Drop Counting Application software subroutine framework, wherein the Automatic Visual Recognition Intravenous Infusion Drop Counting Application software subroutine framework runs on the mobile device and facilitates the administration and accuracy of an intravenous infusion therapy;
computer usable program code configured to determine the occurring drip rate;
computer usable program code configured to input the value of a required drip rate;
computer usable program code configured to compare both, the occurring drip rate detected and a user's required drip rate;
computer usable program code configured to automatically detect the falling drops into a drip chamber;
computer usable program code configured to determine whether or not the occurring drip rate and the required drip rate values are synchronized;
computer usable program code configured to responsively notify the user when the occurring drip rate is synchronized with a required drip rate; and
computer usable program code configured to continue checking the occurring drip rate until the value of the occurring drip rate and the value of the required drip rate are synchronized.

8. A mobile device comprising:
one or more processors;
one or more transceivers for transmitting and receiving wireless data;
one or more sensors for detecting visual events;
one or more storage devices storing program instructions, wherein executing the program instructions by the one or more processes causes the computing device to:
receive at the mobile device an visual activation triggering input for the Automatic Visual Recognition Intravenous Infusion Drop Counting Application software subroutine framework, wherein the Automatic Visual Recognition Intravenous Infusion Drop Counting Application software subroutine framework runs on the mobile device and facilitates the administration and accuracy of an intravenous infusion therapy;
determine an occurring drip rate value associated with the received visual activation input, wherein the Automatic Visual Recognition Intravenous Infusion Drop Counting Application software subroutine framework running in the mobile device use its value to determine if it matches the required drip rate;
capture one or more additional information items to be attached to the infusion therapy tasks and schedules;
automatically displaying the occurring drip rate value;
determine whether or not the process to match the values of the occurring drip rate and the required drip rate has been terminated;
responsively capture one or more updated additional information items to be sent with one or more update emergency messages; and
continue displaying the occurring drip rate until the Automatic Visual Recognition Intravenous Infusion Drop Counting Application software subroutine framework running in the mobile device use its value to determine if it matches the calculated drip rate.

9. The system of claim 8, wherein the Automatic Visual Recognition Intravenous Infusion Drop Counting Application software subroutine framework server maintains patient's profiles, wherein patient's profiles include one or more user information, one or more user specific information.

10. The system of claim 8, wherein the one or more drip rates are previously configured ones which are dependent upon predetermined conditions, wherein the Automatic Visual Recognition Intravenous Infusion Drop Counting Application software subroutine framework executes the visual recognition to compare the given drip rate value versus the occurring drip rate value, wherein said input is from a sensor of the mobile device comprising a camera device.

11. The system of claim 8, wherein the Automatic Visual Recognition Intravenous Infusion Drop Counting Application software subroutine framework improves the accuracy and control of the intravenous infusions given to patients where a infusion pump mechanism is not available.
